SEQUENCE LISTING (30 sequences)

SEQ ID NO: 1 - DNA, 704 bp, Homo sapiens
ggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgcccgaaggacatctaga

SEQ ID NO: 2 - DNA, 718 bp, Homo sapiens
catggcgcacgctgggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgcccgaaggacatctaga

SEQ ID NO: 3 - DNA, 706 bp, Homo sapiens
tgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgcccgaaggacatctaga

SEQ ID NO: 4 - DNA, 708 bp, Artificial Sequence
  Description of Artificial Sequence Synthetic polynucleotide
ggtgcctatctgggccacaagtgaagtcaacatgcctgccccaaacaaatatgcaaaaggttcactaaagcagtagaaataatatgcattgtcagtgatgtaccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgcccgaaggacactcgag

SEQ ID NO: 5 - DNA, 7 bp, Artificial Sequence
  Description of Artificial Sequence Synthetic consensus sequence
ttattta

SEQ ID NO: 6 - DNA, 16 bp, Artificial Sequence
  Description of Artificial Sequence Synthetic consensus sequence
wwttatttattwwwww

SEQ ID NO: 7 - DNA, 42 bp, Homo sapiens
ttattttttacattattaagaaaaaaagatttatttatttaa

SEQ ID NO: 8 - DNA, 70 bp, Mus musculus
tgccaaagggaaatatcatttattttttacattatttaggaaaaaaaagatttatttatttaagacagtt

SEQ ID NO: 9 - DNA, 31 bp, Monodelphis domestica
aaagggaaatatcatttatttttacattatt

SEQ ID NO: 10 - DNA, 45 bp, Mus musculus
tcatttattttttacattattaagaaaaaaagatttatttattta

SEQ ID NO: 11 - DNA, 39 bp, Bos taurus
atattttacattaataaggaaaaaagaattttctattta

SEQ ID NO: 12 - DNA, 475 bp, Bos taurus
ctgggtgcctatctgggccataagtgaagcccacaggcctgccgccaacaaatatgcagaagattcaagaagcagcagaaataatatgcattgttgctgatgcaccactgaagtgaagctgtagtctcttaaaaagaaaaaaaaataaccaaccaatcaaccaacaccaccaccacaaacagctttcaggctcaacgttgaatcagctacttactgccaaagggaaatacaatttattttttacattattaagaaaaataagatttatttatttaacagagtcctgttgaatctgtctttggaaaccccaccacgaattaccaagccccacctggatggctctacctggctctctcgtgcctgtaaacactgatttttgttccatactgtgggctggactgagtcagccagctgacgtgcctgtaaacactgatttttgttccatactgtgggctggactgagtcagccagctga

SEQ ID NO: 13 - DNA, 15 bp, Caenorhabditis elegans
tttatttatttattt

SEQ ID NO: 14 - DNA, 36 bp, Oryza sp.
ataatttattttaaatatttagaaaaaaagatttat

SEQ ID NO: 15 - DNA, 31 bp, Oryza sp.
ggcttttaagtaaaaaaacacatatataaaa

SEQ ID NO: 16 - DNA, 39 bp, Vitis vinifera
attttttacattaatactaaacaaaagatttatttattt

SEQ ID NO: 17 - DNA, 28 bp, Oryza sp.
acattgttaagaaaaaacaattcattta

SEQ ID NO: 18 - DNA, 43 bp, Oryza sp.
tttagttactcctttattaaggataaatagatttatatattta

SEQ ID NO: 19 - DNA, 21 bp, Zea mays
cctttttatttatttatttat

SEQ ID NO: 20 - DNA, 17 bp, Zea mays
atttatttatttattta

SEQ ID NO: 21 - DNA, 16 bp, Zea mays
tatcatttatttttta

SEQ ID NO: 22 - DNA, 17 bp, Arabidopsis thaliana
aagatttatttatttaa

SEQ ID NO: 23 - DNA, 13 bp, Glycine max
atttatttattta

SEQ ID NO: 24 - DNA, 40 bp, Tetrahymena thermophila
attttttactttatttaatataaaaagttttattttttta

SEQ ID NO: 25 - DNA, 41 bp, Dictyostelium discoideum
ataatttattttttaaagtttttagaaataaatatttattt

SEQ ID NO: 26 - DNA, 26 bp, Saccharomyces sp.
attatttaagaaaaaaaattaattaa

SEQ ID NO: 27 - DNA, 26 bp, Saccharomyces sp.
attatttaagaaaaaaaattaattaa

SEQ ID NO: 28 - DNA, 40 bp, Saccharomyces sp.
attcatatttattttatatacaaataaatatatttattta

SEQ ID NO: 29 - DNA, 16 bp, Pichia guilliermondii
aaatatcatttatttt

SEQ ID NO: 30 - DNA, 13 bp, Artificial Sequence
  Description of Artificial Sequence Synthetic consensus sequence
wwatwwwtttaas

(19) United States
(12) Patent Application Publication
(10) Pub. No.: US 2008/0189805 A1
(43) Pub. Date: Aug. 7, 2008

(54) NOVEL GENES AND RNA MOLECULES THAT CONFER STRESS TOLERANCE

(76) Inventors: John J. SERBIN, Del Mar, CA (US); Richard G. SCHNEEBERGER, Carlsbad, CA (US)

Correspondence Address: Biotechnology Law Group;c/o Portfolioip, P.O. Box 52050, Minneapolis, MN, 55402, US

(21) Appl. No.: 11/956,318

(22) Filed: Dec. 13, 2007

Related U.S. Application Data
(60) Provisional application No. 60/874,801, filed on Dec. 13, 2006.

(51) Int. Cl. C12N 15/11; C12N 15/63; C12N 5/10; C12N 1/21; C12N 1/19; A01H 5/00; A01G 16/00; A01G 1/00

(52) U.S. Cl. 800/278; 536/23.1; 435/320.1; 435/419; 435/325; 435/252.3; 435/254.2; 800/298; 800/317.4; 800/317.2; 800/317.3; 800/317.1; 800/314; 800/306; 800/313; 800/305; 800/320; 800/317; 800/312; 800/310; 800/309; 800/307; 800/320.1; 800/320.2; 800/320.3; 800/322; 800/323; 435/468; 47/58.1 R; 47/89

(57) ABSTRACT

Non-coding RNA molecules that include the sequence 5′-UUAUUU-3′, the expression of which confers resistance or tolerance to abiotic and biotic stresses, are described, as are genes encoding the same, expression cassettes and vectors harboring such genes, and transgenic eukaryotic organisms that express such RNAs.

### RELATED APPLICATION

This application claims the benefit of and priority to provisional application Ser. No. 60/874,801 (Attorney docket no. IDV-2001-PV), filed on 13 Dec. 2006, the contents of which are herein incorporated by reference in their entirety for any and all purposes.

### TECHNICAL FIELD

This invention relates generally to genes that encode a novel class of RNA molecules that surprisingly modulate stress resistance or tolerance in eukaryotic organisms, including plants and yeast.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Programmed cell death (“PCD”) and its morphological equivalent, apoptosis, is the active process of genetically controlled cell suicide. PCD has been found to be an intrinsic part of the development, maintenance of cellular homeostasis, and defense against environmental insults, including pathogen attack, in animals. It also plays an essential role in morphogenesis and in development of the immune and nervous systems. Dysregulation of apoptosis, conversely, is involved in the pathogenesis of a number of important diseases in mammals, including cancers, autoimmunity, AIDS, and neurodegenerative disorders.

With recent advances in understanding the complex signaling pathways that induce programmed cell death in animal cells, research has intensified in identifying similar pathways in evolutionarily distant organisms, such as plants. In plants, PCD plays a normal physiological role in a variety of developmental processes, including xylem formation, senescence, sloughing of root cap cells, and embryogenesis. Plant cell death also occurs in response to pathogen challenge, as well as in response to abiotic stresses. Recent evidence suggests that plant cell death might be mechanistically similar to animal apoptosis in some cases such as in plant development, disease associated death, and hypersensitive reaction. The dying plant cells appear morphologically similar to apoptotic cells: they form apoptotic bodies; oligonucleosomal cleavage occurs, often with the characteristics of endonucleolytically processed DNA; and terminal deoxynucleotidyl-transferase-mediated UTP end-labeling has been observed.

Despite these similarities between programmed cell death in plants and animals, some aspects of the function and mechanism of PCD in plants may still differ from what is observed in animals. For example, plant cells do not engulf their dead neighbors, and in some cases, the dead plant cells become part of the very architecture of the plant performing crucial functions such as xylem and phloem. Currently, very little is known about the genes and corresponding proteins that control PCD in plants, and few apoptosis-related animal gene (vertebrate or invertebrate) homologues have been found in detected in plants.

Accordingly, given the recognized importance of apoptosis in animals and the importance of PCD in development and pathogen resistance in plants, understanding analogous plant pathways is extremely valuable, and may lead to methods of regulating the pathway and generating transgenic plants harboring cell death modulators that have unique phenotypic characteristics, such as resistance to various biotic and abiotic insults, as well as increased shelf-life of cut plants, fruits, and vegetables.

The present invention describes the discovery of a novel class of non-coding RNA molecules that, when expressed in plants and yeast, confers protection against a variety of biotic and abiotic stresses. This invention follows a fortuitous discovery made in the course of investigating the effects of expressing various animal anti-apoptotic genes in transformed tobacco plants. Specifically, it was discovered that an untranslated fragment from the 3′-untranslated region (“UTR”) of the human anti-apoptotic bcl-2 gene, serendipitously generated as an unintended cloning artifact, modulates resistance to various biotic and abiotic insults in transgenic plants harboring an expression system that allows transcription of the fragment to yield an untranslated RNA species. At the time of that discovery, nothing was known about the mechanism or attributes that resulted in the observed phenotype. This invention, which excludes that previously discovered singular sequence, concerns a patentable class of RNA molecules capable of conferring resistance or tolerance to abiotic and/or biotic stresses when expressed in eukaryotic cells modified to express such molecules prior to or in response to exposure to one or more stresses, as well as nucleic acid molecules encoding such RNAs, transgenic plants, cells, and tissues that express such RNA molecules, and methods for making an using the same.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “abiotic” insult or stress refers to a plant challenge caused by exposure to a non-viable or non-living agent (i.e., an abiotic agent). Examples of abiotic agents that can cause an abiotic stress include environmental factors such as low moisture (drought), high moisture (flooding), nutrient deficiency, radiation levels, air pollution (ozone, acid rain, sulfur dioxide, etc.), high temperature (hot extremes or heat shock), low temperature (cold extremes or cold shock), and soil toxicity (e.g., toxic levels of salt, heavy metals, etc.), as well as herbicide damage, pesticide damage, or other agricultural practices (e.g., over-fertilization, improper use of chemical sprays, etc.).

A “biotic” insult or stress refers to a plant challenge caused by viable or biologic agents (i.e., biotic agents). Examples of biotic agents that can cause a biotic stress include insects, fungi, bacteria, viruses, nematodes, viroids, mycloplasmas, etc.

A “gene” that codes for a non-coding RNA molecule refers to a nucleic acid molecule that encodes a non-coding RNA, such that expression of that gene results in the synthesis of one or more RNA non-coding RNA molecules therefrom. In some contexts, however, a “gene” may refer to a protein-encoding nucleic acid.

A “host cell” refers to a cell that contains a vector according to the invention.

The terms “include”, “including”, and the like mean “including, without limitation”.

An “isolated nucleic acid molecule” refers to a polynucleotide molecule in the form of a separate fragment or as a component of a larger nucleic acid construct, that has been separated from its source cell (including the chromosome it normally resides in) at least once, and preferably in a substantially pure form. Nucleic acid molecules may be comprised of a wide variety of nucleotides, including deoxyribonucleotides, ribonucleotides, nucleotide analogues in which the pyrimidine or purine base differs from a base that occurs in nature (e.g., adenine, guanine, thymine, cytosine, and uracil) or in which the backbone chemistry linking the various monomers (or dimers or other polymers) differs from the phosphodiester backbone of nucleic acids found in nature, or a combination thereof.

The term “modulate” refers to the ability to alter from a basal level. As used in the context of apoptosis (e.g., to “modulate” apoptosis or PCD), “modulate” refers to the ability to alter or change any biochemical, physiological, or morphological event associated with apoptosis from its basal level. For example, apoptosis has been “modulated” if there has been an alteration in expression of a gene involved in an apoptotic pathway, the interaction of an apoptotic pathway protein with other proteins, the formation of apoptotic bodies, or the DNA cleavage is altered from its original state. Similarly, response to a stress has been “modulated” if, for example, a biochemical, physiological, or morphological parameter (e.g., growth, viability, fruit or send production, photosynthetic rate, rate of respiration or transpiration, etc.) being assessed differs from the level of that parameter in the absence of the stress.

A “non-coding RNA molecule” refers to an RNA molecule that, when expressed in a cell under the control of desired promoter or other element from which transcription can be directed, does not encode a desired polypeptide. Here, “desired polypeptide” refers a protein, peptide, or polypeptide that one intends to express, as opposed to one that is incidentally expressed as the result of an open reading frame that may be translated under some circumstances. In most cases, non-coding RNAs will be encoded by a gene. It will be understood that the nucleic acid molecules of the invention do not include, and indeed, specifically exclude, the fortuitously discovered RNA molecule described in PCT patent application PCT/US2006/004349 (which claims priority to U.S. provisional patent application Ser. No. 60/651,521), which RNA molecule corresponds to the nucleotide sequences set out in SEQ ID NOS (SIDs) 1 and 2 therein and herein (see FIG. 1; it being understood that in an RNA molecule, any “T” would be replaced with “U”).

A “patentable” composition (including plants, plants cells, plant tissues, seeds, protoplasts, etc.), process (or method), machine, or article of manufacture according to the invention means that the subject matter satisfies all statutory requirements for patentability in the particular jurisdiction at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plant pathogen” refers to any agent that causes a disease state in a plant, including viruses, fungi, bacteria, nematodes, and other microorganisms.

A “plant” refers to a whole plant, including a plantlet. Suitable plants for use in the invention include any plant amenable to techniques that result in the introduction of nucleic acid into a plant cell, including both dicotyledonous and monocotyledonous plants. Representative examples of dicotyledonous plants include tomato, potato, arabidopsis, tobacco, cotton, rapeseed, field beans, soybeans, peppers, lettuce, peas, alfalfa, clover, cole crops or Brassica oleracea (e.g., cabbage, broccoli, cauliflower, and Brussels sprouts), radish, carrot, beets, eggplant, spinach, cucumber, squash, melons, cantaloupe, sunflowers, and various ornamentals. Representative examples of monocotyledonous plants include asparagus, field and sweet corn, barley, wheat, rice, sorghum, onion, pearl millet, rye and oat, and ornamentals.

The term “plant cell” refers to a cell from, or derived from, a plant, including gamete-producing cells and cells (e.g., protoplasts) which are capable of regenerating into whole plants. When a cell has been transformed with a nucleic acid or vector according to the invention, it is host cell.

The term “plant tissue” includes differentiated and undifferentiated tissues of a plant, including roots, stems, shoots, leaves, pollen, seeds, tumor tissue, and various forms of cells in culture, including cell suspensions, protoplasts, embryos, and callus tissue.

A “plurality” means more than one.

The term “operably associated” refers to a functional association, or linkage, between a promoter and a gene the expression of which is regulated by the promoter. In the context of this invention, a non-coding RNA is transcribed from, for example, a gene, and the resulting RNA is not translated or used by ribosome as a template for the directing the polymerization of amino acids to form a peptide or polypeptide. In this specification, unless the context otherwise requires, the term “expression” generally refers to the enzyme-mediated transcription of a DNA molecule into an RNA molecule.

A “promoter” refers to a polynucleotide that directs the transcription of a gene operably associated therewith. Typically a promoter is located in the 5′ region of a gene, proximal to the transcriptional start site of a structural gene. A promoter is functional in a eukaryotic cell if it is able to direct expression of the gene(s) operably associated therewith in such cells. A promoter is constitutive if it directs transcription of a gene under most environmental conditions and states of development or cell differentiation. A promoter is inducible if it is capable of directly or indirectly activating transcription of a nucleic acid sequence in response to an inducer. A tissue-specific promoter is a promoter that directs transcription of a gene in a specific plant tissue or tissues. An event specific promoter is a promoter that is active or up-regulated only upon the occurrence of an event, such as exposure to an environmental stress, as a result of viral infection, etc.

The term “transgene” or “heterologous nucleic acid molecule” refers to a nucleic acid molecule containing at least one gene encoding a non-coding RNA species. A heterologous nucleic acid molecule generally, although not necessarily, is a nucleic acid molecule isolated from another species. As will be appreciated, the term “transgene” includes a nucleic acid molecule from the same species, where such molecule has been modified or been placed in operable association with on or more regulatory elements (e.g., a promoter) that differs from the natural or wild-type promoter with which the gene is associated in nature.

A “vector” refers to a DNA or RNA molecule such as a plasmid, cosmid, bacteriophage, or other viral genome that has the capability of replicating in a host cell, and includes cloning vectors, shuttle vectors, and expression vectors. A “cloning” or “shuttle” vector typically contains one or several restriction endonuclease recognition sites into which foreign or heterologous DNA molecules can be inserted in a determinable fashion without loss of essential biological function of the vector, as well as a marker gene that encodes a gene product useful for the identification and selection of cells transformed with the vector. An “expression vector” is typically a DNA molecule (although RNA viral genomes may also be used) that includes at least one gene the expression of which is desired in a host cell. Typically, the expression of the gene(s) introduced into the vector for expression is under the control of one or more regulatory elements suitable for use in the intended host cell. Such regulatory elements include enhancers, promoters, termination signals, and polyadenylation sites.

A “wild-type” plant or plant variety refers to a plant that does not contain a transgene or nucleic acid according to the invention. As such, the plant may, in fact, be a transgenic plant, although any transgene(s) contained in such “wild-type” plant will comprise a nucleic acid other than a nucleic acid according to this invention.

### SUMMARY OF THE INVENTION

The present invention concerns patentable non-coding RNAs (i.e., RNAs that are not translated, except incidentally, if at all) that provide protection against stress when expressed in cells harboring a gene (generally a transgene) encoding the non-coding RNA molecule, as well as eukaryotic organisms, such as yeast and transgenic plants and plant cells, tissues, and products that express an untranslated RNA molecule from a corresponding transgene. In general, the non-coding RNA minimally comprises the sequence 5′-UUAUUUA-3′. Two or more copies of this sequence may also be present in any such sequence. Genes encoding such RNA molecules will be referred to herein as comprising the corresponding sequence 5′-UUATTTA-3′. Preferred embodiments o such sequences correspond to the DNA sequences set out in each of SIDs 5-30. It is understood, however, that the non-coding RNA molecule may also be expressed as part of an RNA that also includes a coding region, such as a naturally occurring or non-naturally occurring peptide or polypeptide. Preferably, if the peptide or polypeptide is a naturally occurring, it contains fewer than all of the amino acid residues found in the naturally occurring molecule.

Thus, one aspect of the invention relates to transgenic organisms, including transformed yeasts and plants and plant cells, tissues, and products derived therefrom, while two other aspects concern the non-coding RNA molecules themselves and the genes encoding them.

Related aspects concern expression cassettes that comprise a promoter operably associated with a gene encoding a non-coding RNA molecule of the invention, as well as vectors that include such molecules, particularly expression vectors that include an expression cassette of the invention. In some embodiments, the expression cassette may further comprise a second nucleic acid molecule that encodes an expression product that confers a second desired trait, such as resistance to an insect pest (as may be achieved, for example, by the expression in the plant, or selected cells or tissues thereof, of a toxin that kills an insect pest that preys upon the particular plant species) and/or resistance to an herbicide (for example, glyphosate). Expression of the desired transgene(s) is under the control of a promoter, a constitutive promoter or an inducible promoter.

A related aspect concerns host cells, for example, mammalian cells, yeast cells, plant cells, and bacterial cells, transformed with a vector of the invention.

Preferably, the transgenic cells and organisms of the invention exhibit increased stress resistance or tolerance when cultivated under stressful conditions, as compared to a wild-type plant of the same variety as the transgenic plant. Transgenic plants of the invention may also have one or more tissues that exhibit reduced senescence, as compared to the same tissue(s) of a wild-type plant of the same variety as the transgenic plant.

Representative plants that can be transformed with the nucleic acids of the invention include tomato, potato, arabidopsis, tobacco, cotton, rapeseed, field bean, soybean, pepper, lettuce, pea, alfalfa, clover, cole, cabbage, broccoli, cauliflower, Brussels sprout, radish, carrot, beet, eggplant, spinach, cucumber, squash, melon, cantaloupe, sunflower, ornamental, asparagus, corn, barley, wheat, rice, sorghum, onion, pearl millet, rye, and oat plants.

The invention also concerns various methods, including those for producing transgenic plant and yeast cells, for example, by transforming a eukaryotic cell with a gene, preferably in a an expression cassette carried on a vector. In the case of cells from multicellular organisms, such organisms, for example, a transgenic plant, may then be generated. Given their improved stress resistance or tolerance, the transgenic organisms of the invention can be cultivated environments where they may be exposed under anticipated conditions to a stress which, in the absence of expression of a non-coding RNA molecule of the invention, would result in injury to or death of the cells, tissue, and/or organism.

### BRIEF DESCRIPTION OF THE FIGURES

FIG. 1 shows an alignment of four polynucleotide sequences (SEQ ID NOS, or “SIDS”, 1-4). Two of the aligned sequences (SIDs 1 and 2) represent different versions of the nucleic acid sequence that that encodes the fortuitously discovered untranslated fragment from the 3′-untranslated region (“UTR”) of the human anti-apoptotic bcl-2 gene, each of which is specifically excluded from the scope of this invention. The two versions of the sequence differ only in that the upper sequence in the alignment contains an additional 14 bases at the 5′-end (SEQ ID NO: 2; SID 2), as compared to the other version (SEQ ID NO: 1; SID 1), due to the inclusion of several bases in SEQ ID NO: 2 from a cloning site in a vector. SID 3 represents the corresponding region of the 3′-UTR of the human bcl-2 gene. SID 4 represents a preferred sequence of the invention. In the alignment, “-” represents a gap or missing base in a comparison of sequence to another. The asterisks below the alignment serve to highlight the positions where there is a difference between at least two of the sequences in the alignment at the given positions.

FIG. 2 is a table listing various preferred embodiments of the invention.

FIG. 3 is a diagrammatic representation illustrating various elements that can be included in an expression cassette for expressing a stress protection sequence (STS) according to the invention. Here, the STS is represented by the designation “ARE Seq”.

FIG. 4 shows a diagrammatic representation of several vectors of the invention, as described in Example 2, below.

FIG. 5 has two panels, A and B, illustrating some of results from the experiments described in Example 2, below. Specifically, panel 5A is a plot of cell culture OD₆₀₀ versus time. Error bars are included for all time points, and are equal to the standard error of the mean (calculated using mean and standard deviation of eight biological replicates). Empty vector=control yeast strain:plasmid and iDi−176=test yeast strain:plasmid). Panel 5B is a plot of log OD₆₀₀ versus time.

FIG. 6 has two panels, A and B, illustrating some of results from the experiments described in Example 2, below. Specifically, panel 6A is a plot of colony forming units per milliliter of culture medium vs. time after initiating hydrogen peroxide stress. Error bars are included for all time points, and are equal to the standard error of the mean (calculated using mean and standard deviation of eight biological replicates). Empty vector=control yeast strain:plasmid and iDi−176=test yeast strain:plasmid. Panel 6B is a log plot of the results shown in panel 6A.

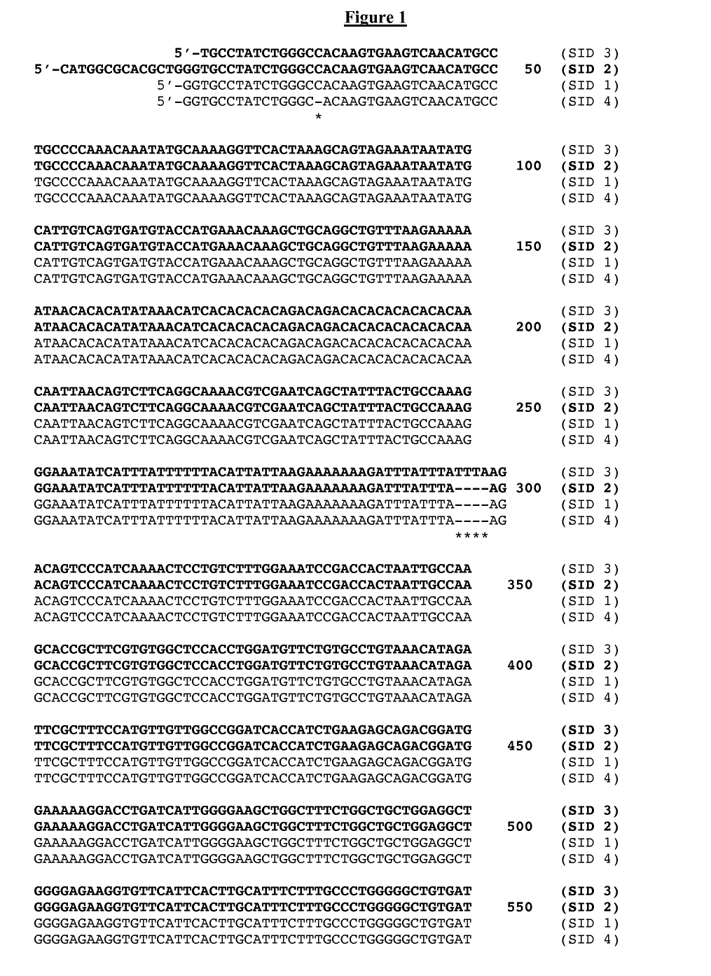

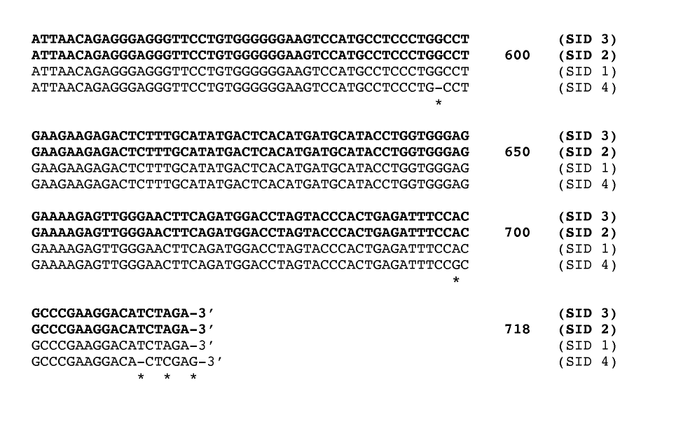

### DETAILED DESCRIPTION

The present invention is based on the discovery of a novel class of non-coding RNA molecules, and genes encoding them, which, when expressed in eukaryotic cells, confer resistance or tolerance to one or more biotic and abiotic stresses. In general, the RNA molecules of the invention comprise at least one copy of the following nucleotide sequence: 5′-AUUUA-3′ (and genes encoding such RNAs include the corresponding nucleotide sequence: 5′-ATTTA-3′). These RNAs may derived from natural sources or they may be wholly or partially synthetic. These RNA molecules, nucleic acids encoding them, vectors that contain such RNA-encoding nucleic acids, eukaryotic cells transformed with such nucleic acids, and methods for making and using the same, are described in detail below.

### 1. Nucleic Acids

The non-coding RNA molecules of the invention are those that confer resistance or tolerance to one or more biotic and abiotic stresses when expressed from a transgene encoding them, but they specifically exclude those that correspond to the nucleobase sequence set out in SEQ ID NO: 1 or 2, below. Such molecules can be identified using any suitable screening method, and once identified, stress tolerance activity can be confirmed by any suitable assay (for example, by comparing a population of cells (e.g., a yeast cell) genetically modified to express a particular RNA molecule of the invention versus a wild-type or otherwise similar but unmodified form of the same organism under abiotic or biotic stress. Those RNA molecules that confer the desired tolerance or stress can then be used in accordance with other aspects of the invention.

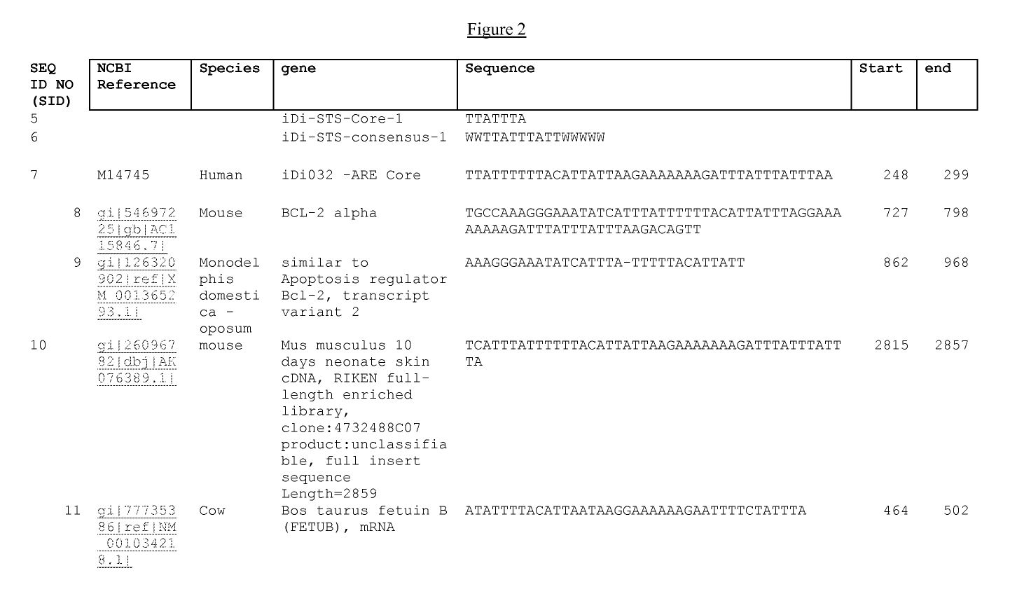

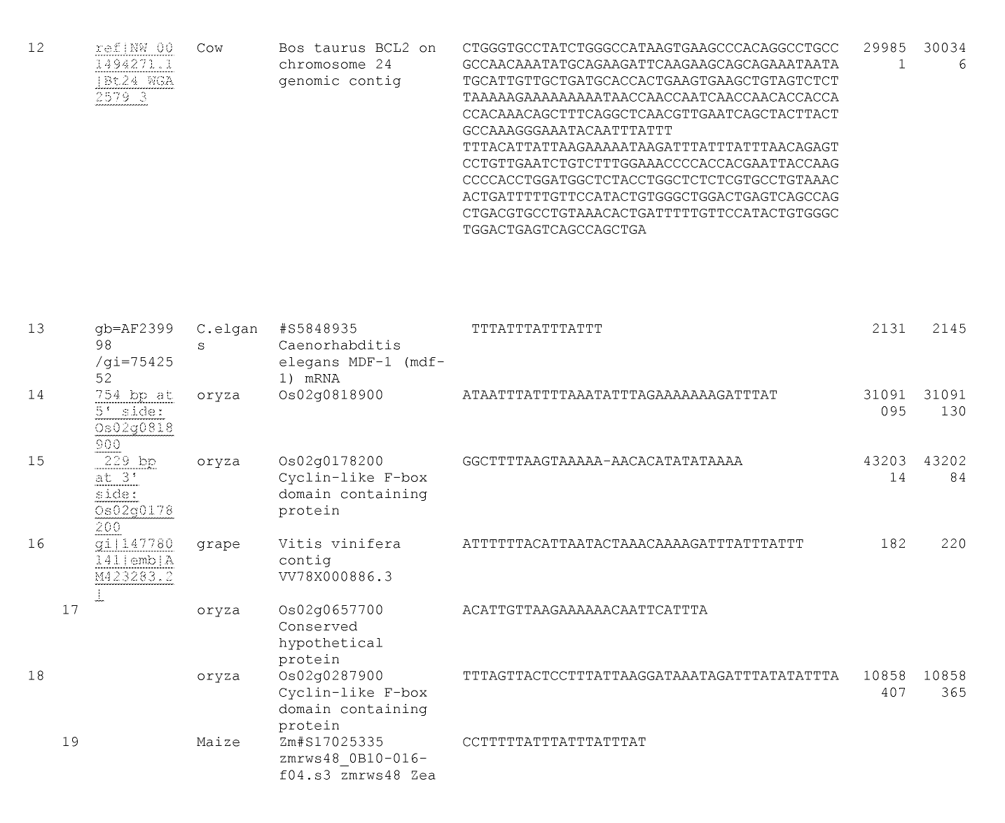

A preferred sub-class of the RNA molecules of the invention are those that include adenylate uridylate-rich elements (AREs) represented by the sequence 5′-UUAUUUA-3′ and the corresponding DNA sequence 5′-TTATTTA′-3 (referred to as the “iDi-STS-Core-1” sequence in FIG. 2). Other preferred embodiments include RNA molecules that include as a core STS sequence that corresponds to an encoding DNA having the sequence 5′-TTATTTATT′-3,5′-WWTTATTTATTWWWWW-3′ (where “W” can be A or T), 5′-WWATWWWTTTAAS-3′ (where “S” can be G or C), or any of SIDs 7-29.

The RNA molecules of the invention include at least one such sequence. Preferably, such RNA molecules, and the DNA molecules that encode them (here, a “gene”, although in the context of the invention such a “gene” may not, and in many preferred embodiments, does not, include a region that encodes a peptide, polypeptide, or protein intended to be expressed) for the expression of any amino acid residue) contain about 25 to about 10,000 nucleotides. Preferred RNAs range from about 40 to less than about 2,000 bases, preferably from about 50 to less than about 1,500, 1,000, 750, or 500 bases.

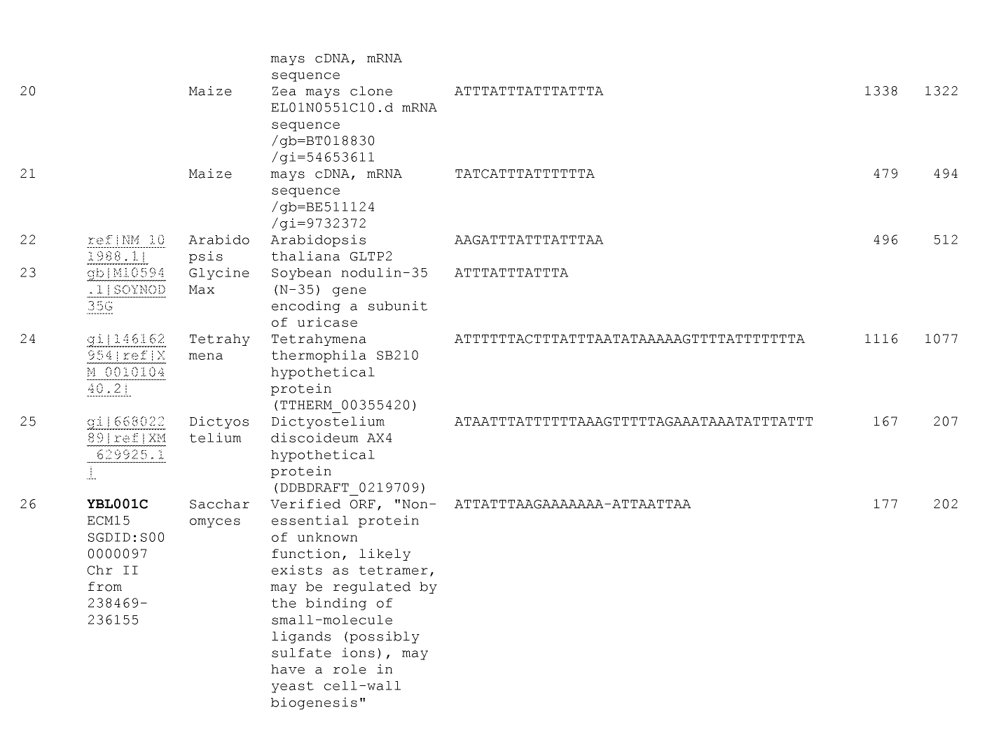

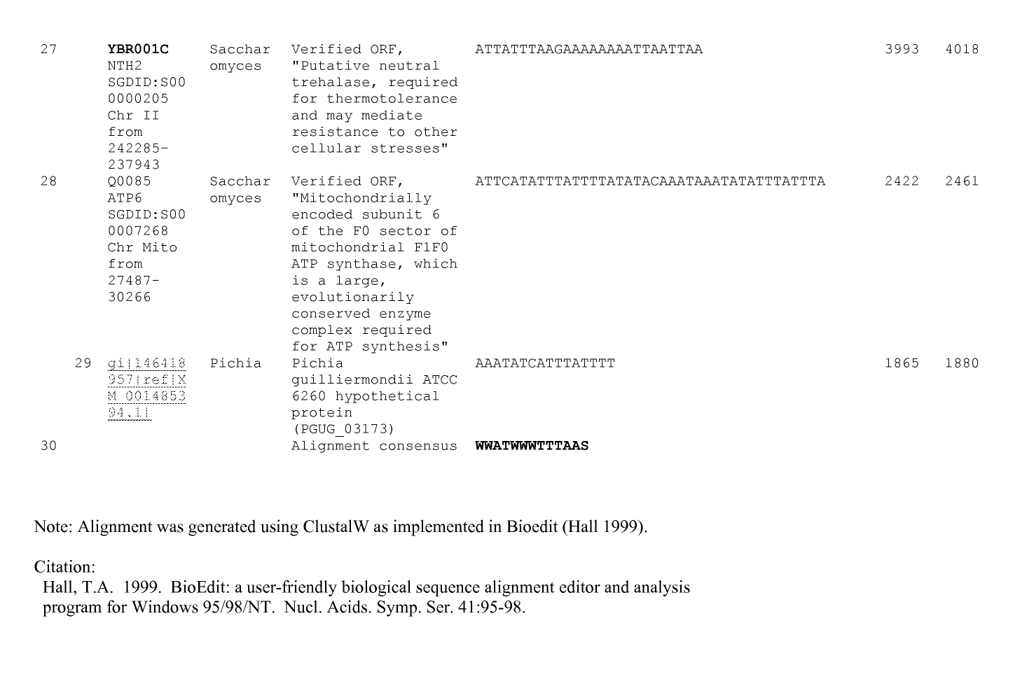

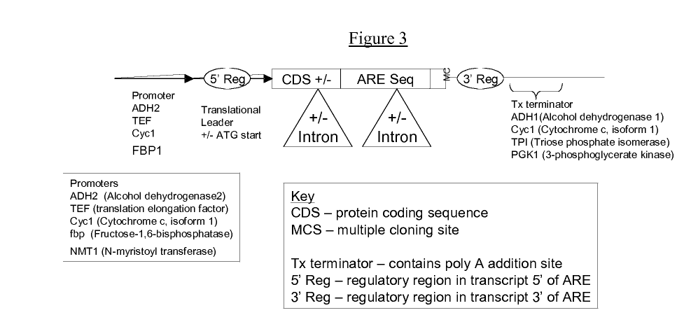

The RNA molecules of the invention, and the DNAs or expression cassettes that encode them, can include a variety of elements in addition to an STS (stress protection sequence). Examples of such other elements are indicated in FIG. 3, and include translational leader sequences that may or may not encode a peptide or series of amino acids, peptide or polypeptide-encoding sequences, intron splice sites (which can serve, for example, to facilitate proper mRNA processing and transport from the nucleus), sequences to facilitate cloning, and regulatory sequences (e.g., terminator sequences, polyadenylation sequences, etc.).

AREs have been reported to in animals, particular mammals such as humans. Current understanding provides that AREs mediate the rapid turnover in cis of mRNAs encoding a wide repertoire of functionally diverse proteins that regulate cellular growth and body response to exogenuous agents such as microbes, as well as to inflammatory and environmental stimuli. In the context of the invention, however, it has been discovered that non-coding sequences that contain one or more AREs may also function in trans. Without wishing to be bound a particular theory, it is believed that ARE-containing non-coding RNAs affect proteosome function. Specifically, using a yeast three-hybrid system, a tomato protein, Ubiquitin-conjugating enzyme E2, was identified that interacts with an ARE-containing non-coding RNA. This result was confirmed by gel electrophoresis mobility shift assays. Using the tomato E2 protein as bait and tomato cDNA library as prey, yeast two-hybrid screening was then used to determine that E2 interacted with ubiquitin and Q5, a Z-finger protein. Q5 contains two z-finger domains: ZnF-A20-(an inhibitor of cell death)-like zinc finger and AN1-(an ubiquitin-like protein)-like zinc finger. Together, this data indicates that non-coding RNA molecules of the invention may mediate their stress-protective effects by modulating specific protein degradation via the ubiquitin/proteasome pathway. Further evidence for the involvement of non-coding RNA molecules and proteasomes are that E2 and Q5 inhibit cell death induced by H₂O₂, heat shock, and Bax when expressed in yeast engineered to express a non-coding RNA molecule. When the E2 and Q5 genes are expressed in an E1 deletion mutant yeast strain, however, neither the non-coding RNA, E2, nor Q5 were unable to confer stress protection against H₂O₂ or heat.

The transgenes that encode the non-coding RNA molecules of the invention can be single- or double-stranded. For purposes of this invention, a gene that “consists essentially of” a particular sequence minimally includes polymerized nucleotides having that sequence, alone or having one or more nucleotides added to either or both the 5′ and/or 3′ ends of the molecule, provided that such additional nucleotides do not materially alter the stress-tolerating function of the non-coding RNA species transcribed from the DNA molecule. Similarly, a gene “consists of” a particular sequence when it includes polymerized nucleotides encoding only that sequence.

Nucleic acids according to the invention or fragments thereof (including those made by various synthetic techniques) may be used as probes for screening to confirm transformation, determine copy number or level of expression of the transgene, etc. To facilitate hybridization-based detection, such probes may be labeled with a reporter molecule, such as a radionuclide (e.g., ³²P, ³⁵S, etc.), enzymatic label, protein label, fluorescent label, biotin, or other detectable moiety. Alternatively, nucleic acid amplification-based techniques known in the art (e.g., PCR, transcription-mediated amplification, strand-displacement amplification, etc.) may be readily adapted for such purposes through the design and use of suitable primers.

### 2. Vectors Host Cells and Transgene Expression

The present invention encompasses vectors comprising regulatory elements operably associated with a nucleic acid molecule encoding a non-coding RNA molecule of the invention. Such vectors may be used, for example, in the propagation and maintenance of nucleic acid molecules of the invention, or in the expression and production of RNA transcripts from such nucleic acid molecules. Depending upon the intended use, those skilled in the art can select any suitable vector. Suitable vectors include plasmids, cosmids, episomes, and viral genomes, including those adapted for gene transfer from baculovirus, retrovirus, lentivirus, adenovirus, and parvovirus.

Nucleic acid molecules of the invention may be expressed in a variety of host organisms, including mammalian cells (e.g., CHO, COS-7, and 293 cells), other eukaryotes such as yeast (e.g., Saccharomyces cerevisiae) and insect cells (e.g., Sf9), as well as bacterial cells (e.g., E. coli and Bacillus). Expression of an instant nucleic acid in, for example, the context of the production of a recombinant protein (e.g., an antibody, a growth factor, a hormone, an enzyme, etc.) can be used to increase the yield of the desired protein product. In other particularly preferred embodiments, a nucleic acid molecule according to the invention is expressed in plant cells. Vectors suitable for use with any of these host cells are well known in the art.

In preferred embodiments, a DNA molecule of the invention is introduced into a vector to form an expression cassette. The DNA molecule is derived from an existing clone or synthesized. Preferred synthetic routes include nucleic acid-based amplification (e.g., PCR) of a structural gene of the invention. Such gene may be present, for example, in cDNA, genomic DNA, or in a recombinant clone. Amplification is performed using a set of primers that flank the structural gene. Restriction sites are typically incorporated into the primer molecules to facilitate subsequent cloning steps, and should be chosen with regard to the cloning site of the vector. If desired, termination signals, polyadenylation signals, etc. can also be engineered into an amplification primer.

At minimum, the expression cassette vector will also contain a promoter. The promoter will contain an RNA polymerase binding site, and, in eukaryotes, promoters frequently contain binding sites for other transcriptional factors that control the rate and timing of gene expression. Such sites include the so-called TATA box, CAAT box, POU box, API binding site, and the like. Promoter regions may also contain enhancer elements. The promoter may be in any suitable form. Depending upon the intended application, promoters may provide for constitutive or inducible expression of the nucleic acid molecule of the invention, as desired in the particular system.

The expression cassettes of the expression vectors of the invention include a promoter designed for expression of a structural gene according to the invention. Such promoters for expression in bacteria include promoters from the T7 phage and other phages, such as T3, T5, and SP6, and the trp, lpp, and lac operons. Hybrid promoters (see, e.g., U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. U.S. Pat. No. 4,870,009), 35S promoter of CaMV, alcohol dehydrogenase gene promoter, chitinase gene promoter, and the like.

The promoter that controls transcription of a gene according to the invention may itself be controlled by a repressor. In some systems, the promoter can be derepressed by altering the physiological conditions of the cell, for example, by the addition of a molecule that competitively binds the repressor, or by altering the temperature of the growth media. Preferred repressors include the E. coli lacI repressor responsive to IPTG induction, the temperature sensitive lambda cI857 repressor, and the like.

In other preferred embodiments, the vector also includes a transcription terminator sequence. A “transcription terminator region” has either a sequence that provides a signal that terminates transcription by the RNA polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the f1-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host cell into which the vector is introduced. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}), and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance).

One skilled in the art appreciates that there are a wide variety of suitable vectors for expression in bacterial cells that are readily obtainable. Vectors such as the pET series (Novagen, Madison, Wis.), the tac and trc series (Pharmacia, Uppsala, Sweden), pTTQ18 (Amersham International plc, England), pACYC 177, the pGEX series, and the like are suitable for expression of BAG-1. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see, e.g., U.S. Pat. No. 4,745,051). As will be appreciated, different vectors are paired with suitable hosts.

A wide variety of suitable vectors for expression in eukaryotic cells are also available. Such vectors include pCMVLacI and pXT1 available from Stratagene Cloning Systems (La Jolla, Calif.), and pCDNA series, pREP series, and pEBVHis available from Invitrogen (Carlsbad, Calif.). In certain embodiments, a BAG nucleic acid molecule is cloned into a gene targeting vector, such as pMC1 neo and a pOG series vector (Stratagene Cloning Systems).

The invention also includes as preferred embodiments plant vectors into which a nucleic acid molecule according to the invention has been inserted. General descriptions of plant expression vectors and reporter genes can be found in Gruber, et al., “Vectors for Plant Transformation, in Methods in Plant Molecular Biology & Biotechnology” in Glich, et al., Eds. pp. 89-119, CRC Press, 1993. Moreover, GUS expression vectors and GUS gene cassettes are available from Clontech Laboratories, Inc. (Palo Alto, Calif.), while GFP expression vectors and GFP gene cassettes are available from Aurora Biosciences (San Diego, Calif.).

The introduction of a vector into various cells, such as bacterial, yeast, insect, mammalian, and plant cells, are well known. For example, a vector can be transformed into a bacterial cell by heat shock, electroporation, or any other suitable technique. Transformation of yeast cells with a vector according to the invention may also be carried out by electroporation, for example. Methods for introduction of vectors into animal cells include calcium phosphate precipitation, electroporation, dextran-mediated transfection, liposome encapsulation, nucleus microinjection, and viral or phage infection. The introduction of heterologous nucleic acid sequences into plant cells can be achieved by particle bombardment, electroporation, microinjection, and Agrobacterium-mediated gene insertion (for reviews of such techniques, see, e.g., Weissbach & Weissbach, Methods for Plant Molecular Biology, Academic Press, NY, Section VHI, pp. 421-463; 1988; Grierson & Corey, Plant Molecular Biology, 2d Ed., Blackie, London, Ch. 7-9, 1988; and Horsch, et al., Science, vol. 227:1229, 1985; and Gene Transfer to Plants, eds. Potrykus. Springer Verlaag, 1995).

### 3. Transgenic Cells and Organisms

As described above, a primary aspect of this invention concerns transgenic cells and organisms, particularly eukaryotic cells (e.g., animal cells such as mammalian or insect cells, yeast cells, etc.) used for the bioproduction of commercially important intermediates or end products, as well as plants, that are resistant to or tolerant of one more abiotic and/or biotic stresses as a result of the expression of one or more non-coding RNA molecule species of the invention.

A. General Methods

Generally, a transgenic plant is generated by (a) transforming a plant cell with a nucleic acid of interest and (b) regenerating the plant cells to provide a differentiated plant. Frequently, resulting transgenic plants are examined to confirm the presence of the desired transgene. The nucleic acid of interest is usually contained in a vector. However, naked nucleic acid of interest may also be used even though only low efficiency transformation will likely occur.
1. Vectors and Expression Cassettes

Although a general discussion of vectors of this invention is provided above, the following description contains additional information specific to vectors useful in plant cell transformation. Usually, to be effective in regulating the expression, a promoter functional in the plant cells to be transformed is operably associated with a nucleic acid molecule of the invention to form an expression cassette that is carried in the vector. Additionally, a polyadenylation sequence and/or transcription control sequence, also recognized in plant cells, may also be included in the expression cassette in operable association with the promoter and structural gene. It is also preferred that the vector contain one or more genes encoding selectable markers so that transformed cells can easily be selected from non-transformed cells in culture.
(a) Promoters

Any promoter functional in plant cells may be used for generating transgenic plants of this invention, including constitutive, inducible/developmentally regulated, and tissue-specific promoters. Although endogenous plant promoters and the human bcl-2 promoter may be utilized in some embodiments, preferably the promoters are heterologous to the structural gene. Such regulatory sequences may be obtained from plants, viruses or other sources.

Examples of constitutive promoters include the 35S RNA and 19S RNA promoters of cauliflower mosaic virus (CaMV), the promoter for the coat protein promoter to TMV (Akamatsu, et al., EMBO J. 6:307, 1987), promoters of seed storage protein genes such as Zma10Kz or Zmag12 (maize zein and glutelin genes, respectively), “housekeeping genes” that are express in some or all cells of a plant, such as Zmaact, a maize actin gene (see Benfey, et al., Science, vol. 244:174-181, 1989; Elliston in Plant Biotechnology, eds. Kung and Arntzen, Butterworth Publishers, Boston, Mass., p. 115-139, 1989), the patatin gene promoter from potato (see, e.g., Wenzler, et al., Plant Mol. Biol., vol. 12:41-45, 1989), the ubiquitin promoter (see, e.g., EP Patent Application 0342926), and the Chlorella virus DNA methyltransferase promoter (see, e.g., U.S. Pat. No. 5,563,328)

Inducible promoters are also useful in practicing the present invention. An inducible promoter is capable of directly or indirectly activating transcription of an operably associated nucleic acid molecule in response to an inducer. The inducer may be biotic or abiotic, such as a light, heat, cold, a protein, a metabolite (sugar, alcohol, etc.), a growth regulator, a herbicide, etc., or indirectly through the action of a pathogen or disease agent such as a virus. A plant cell containing an inducible promoter may be exposed to an inducer by externally applying the inducer to the cell such as by spraying, watering, heating, exposure to light, exposure to a pathogen, or similar methods.

To be most useful, an inducible promoter preferably provides low or no expression in the absence of the inducer; provides high expression in the presence of the inducer; and uses an induction scheme that does not interfere with the normal physiology of the plant and has little effect on the expression of other genes. Examples of inducible promoters useful within the context of the present invention include those induced by chemical means, such as the yeast metallothionein promoter activated by copper ions; In2-1 and In2-2 regulator sequences activated by substituted benzenesulfonamides, e.g., herbicide safeners; the promoter sequence isolated from a 27 kD subunit of the maize glutathione-S-transferase (GST II) gene induced by N,N-diallyl-2,2-dichloroacetamide (common name: dichloramid) or benzyl-2-chloro-4-(trifluoromethyl)-5-thiazolecarboxylate (common name: flurazole); GRE regulatory sequences induced by glucocorticoids, and an alcohol dehydrogenase promoter induced by ethanol. Other inducible promoters include those induced by pathogen attack (see, e.g., U.S. Pat. No. 6,100,451), a chalcone synthase promoter, and the defense activated promoter (prop1-1) (Strittmatter, et al., Bio/Technology, vol. 13:1085-1089, 1995). Inducible promoters also the inducible promoters from the PR protein genes, especially the tobacco PR protein genes, such as PR-1a, PR-1b, PR-1c, PR-1, PR-A, PR-S, the cucumber chitinase gene, and the acidic and basic tobacco beta-1,3-glucanase genes. Wound inducible (WIN) promoters may also be useful in the context of the present invention.

Tissue-specific promoters may also be utilized. Specific examples of tissue-specific promoter include shoot meristem-specific promoters; the tuber-directed class I patatin promoter; promoters associated with potato tuber ADPGPP genes; the seed-specific promoter of beta-conglycinin, also known as the 7S protein; seed-specific promoters from maize zein genes; pollen-specific promoters (see, e.g., U.S. Pat. Nos. 5,086,169 and 5,412,085); an anther-specific promoter (see, e.g., U.S. Pat. No. 5,477,002); and a tapetum-specific promoter (see, e.g., U.S. Pat. No. 5,470,359).
(b) Markers

The vectors of the present invention, also preferably include at least one selectable or scorable marker/reporter that is functional in plant cells. A selectable marker gene includes any gene that confers a phenotype or trait on the host cells that allows transformed cells to be identified and selectively grown. Accordingly, the selection marker genes may encode polypeptides that confer on plant cells resistance to a chemical agent or to physiological stress, or a distinguishable phenotypic characteristic to the cells such that plant cells transformed with the recombinant nucleic acid molecule may be easily selected using a selective agent. Specific examples for the genes suitable for this purpose have been identified may be found in, for example, Fraley, in Plant Biotechnology, eds. Kung and Amtzen, Butterworth Publishers, Boston, Mass., p. 395-407, 1989, and in Weising, et al., Ann. Rev. Genet., vol. 22:421-77, 1988.
2. Transformation

Plant cell transformation may be carried out using any suitable technique for introducing nucleic acids into plant cells. See, e.g., Methods of Enzymology, vol. 153, 1987, Wu and Grossman, Eds., Academic Press). Herein, “transformation” means alteration of the genotype of cell by the introduction of one or more heterologous nucleic acid molecules. Transformation may be either transient or permanent, with permanent genetic alteration being preferred.

Methods of introducing vectors into monocotyledenous or dicotyledenous plant cells include physical and/or chemical means, such as electroportation, microinjection into plant cell protoplasts, particle bombardment, and viral and bacterial infection/co-cultivation. and are applicable to both monocotyledenous and dicotyledenous plants. The principle methods of causing stable integration of exogenous DNA into plant genomic DNA include the following approaches: (1) Agrobacterium-mediated gene transfer (see, e.g., Horsch, et al., Science, vol 227:1229, 1985; Klee, et al., Annu. Rev. Plant Physiol., vol. 38:467-486, 1987; Klee, et al., Mol. Bio. Of Plant Nucl. Genes, vol. 6:2-25, 1989; Gatenby, Plant Biotech., vol. 93-112, 1989; White, Plant Biotech., vol. 3-34 1989; (2) direct DNA uptake (see, e.g., Paszkowski, et al., Mol. Bio. of Plant Nucl. Genes, vol. 6:52-68, 1989), including methods for direct uptake of DNA into protoplasts (see, e.g., Toriyama, et. al., Bio/Technology, vol. 6:1072-1074, 1988); DNA uptake induced by brief electric shock of plant cells (see, e.g., Zhang, et al., Plant Cell Rep. 7:379-384, 1988, and Fromm, et al., Nature, vol. 319:791-792, 1986); DNA injection into plant cells or tissues by particle bombardment (see, e.g., Klein, et al., Progress in Plant Cellular and Molecular Biology, 56-66, 1988, Klein, et al., Bio/Technology, vol. 6:559-563, 1988, McCabe, et al., Bio/Technology, vol. 6:923-926, 1988, and Sanford, Physiol. Plant, vol. 79:206-209, 1990); by the use of micropipette systems (see, e.g., Hess, Int. Rev. Cytol, vol. 107:367-395, 1987, Neuhaus, et al., Theor. Appl Genet., vol. 75:30-36, 1987, Neuhaus and Spangenberg, Physiol. Plant., vol. 79:213-217, 1990); and by the direct incubation of DNA with germinating pollen, DeWet, et al., Experimental Manipulation of Ovule Tissue, 197-209, 1985, Ohta, Y., Proc. Natl. Acad. Sci USA, vol. 83:715-719, 1986; or (3) the use of a plant virus as a vector (see, e.g., Klee, et al., Ann. Rev. Plant Physiol., vol. 38:467-486, 1987; Futterer, et al., Physiol. Plant, vol. 79:154-157, 1990; and U.S. Pat. Nos. 5,500,360; 5,316,931, and 5,589,367). As those in the art will appreciate, the particular transformation method chosen will depend on many factors, including the species of the plant cells to be transformed, but in any event is a matter of routine.

It may be useful to generate a number of individual transformed plants with any recombinant construct in order to recover plants free from any effects related to the position in which the expression cassette becomes integrated. In certain embodiments it may be preferable to select plants that contain one copy of the introduced nucleic acid molecule, while in other embodiments, multiple copies of the expression may be preferred.

In particularly preferred embodiments, the Agrobacterium Ti plasmid system is utilized to perform plant cell transformation. The tumor-inducing (Ti) plasmids of A. tumefaciens contain a segment of plasmid DNA known as transforming DNA (T-DNA) that is transferred to plant cells where it integrates into the plant host genome. The construction of the transformation vector system typically has two basic steps. First, a plasmid vector is constructed that replicates in E. coli. This plasmid contains an expression cassette capable of directing the expression of a DNA molecule according to the invention (e.g., a DNA having a nucleotide sequence of SEQ ID NO: 1 or 2) flanked by T-DNA border sequences that define the points at which the DNA integrates into the plant genome. Usually a gene encoding a selectable marker (such as a gene encoding resistance to an antibiotic such as Kanamycin) is also inserted between the left border (LB) and right border (RB) sequences. The expression of this gene in transformed plant cells allows for positive selection of plant cells that contain an integrated T-DNA region. The second step entails transfer of the plasmid from E. coli to Agrobacterium. This can be accomplished via a conjugation mating system, or by direct uptake of plasmid DNA by Agrobacterium. For subsequent transfer of the T-DNA to plants, the Agrobacterium strain utilized contains a virulence (vir) genes for T-DNA transfer to plant cells. Those skilled in the art recognize that there are multiple choices of Agrobacterium strains and plasmid construction strategies that can be used to optimize genetic transformation of plants. Methods of inoculation of the plant tissue vary depending upon the plant species and the Agrobacterium delivery system. A very convenient approach is the leaf disc procedure that can be performed with any tissue explant that provides a good source for initiation of whole plant differentiation. The addition of nurse tissue may be desirable under certain conditions. Other procedures such as the in vitro transformation of regenerating protoplasts with A. tumefaciens may be followed to obtain transformed plant cells as well.

In other embodiments, transformation is accomplished using direct physical or chemical means. For example, the nucleic acid can be physically transferred by microinjection directly into plant cells by use of micropipettes or particle bombardment. Alternatively, the nucleic acid may be transferred into the plant cell by using polyethylene glycol which forms a precipitation complex with genetic material that is taken up by the cell (Paszkowske, et al., Proc. Natl Acad. Sci., USA, vol. 82:5824, 1985).

Another method for introducing nucleic acid into a plant cell is high velocity ballistic penetration by small particles that either contain or are coated with the nucleic acid to be introduced (see, e.g., U.S. Pat. Nos. 4,945,050, 5,036,006, and 5,100,792). Typically, when utilizing particle bombardment, the DNA to be delivered is adsorbed on microprojectiles such as magnesium sulfate crystals or tungsten particles, and the microprojectiles are physically accelerated into cells or plant tissues.

Heterologous nucleic acid can also be introduced into plant cells by electroporation. In this technique, plant protoplasts are electroporated in the presence of vectors or expression cassettes containing a nucleic acid molecule according to the invention. Electrical impulses of high field strength reversibly permeabilize membranes allowing the introduction of the nucleic acids into the plant cells. Electroporated plant protoplasts reform cell walls, divide, and form callus tissue. Selection of transformed plant cells can be accomplished using any suitable technique.

After selecting transformed cells, expression of the desired untranslated RNA can be confirmed. For example, simple detection of RNA transcribed from the inserted DNA can be achieved by well-known methods in the art, such as Northern blot analysis. Alternatively, the inserted sequence can be identified, for example, using the polymerase chain reaction and Southern blot analysis. Expression levels and copy number can also be assessed using well-known techniques.
3. Regeneration of Transgenic Plants

Transformed plant cells that express a desired untranslated RNA species can be regenerated into a whole plant using any known technique. Here, “regeneration” refers to growing a whole plant from a transformed protoplast, a plant cell, a group of plant cells (e.g., plant callus), a plant tissue, or a plant organ or part.

Regeneration from protoplasts varies from species to species of plants, but generally a suspension of protoplasts is first made. In certain species, embryo formation can then be induced from the protoplast suspension, to the stage of ripening and germination as natural embryos. The culture media generally contains various amino acids and hormones necessary for growth and regeneration. Examples of hormones utilized include auxin and cytokinins. It is sometimes advantageous to add glutamic acid and proline to the medium, especially for such species as corn and alfalfa. Efficient regeneration depends on many variables, including the medium used, the genotype of the plant cells, and the history of the culture.

Regeneration also occurs from plant callus, tissues, organs, or parts. Transformation can be performed in the context of organ or plant part regeneration (see, e.g., Methods in Enzymology, vol. 118, and Klee, et al., Ann. Rev. Plant Phys., vol. 38:467, 1987). Utilizing a leaf disk-transformation-regeneration method (see, e.g., Horsch, et al., Science, vol. 227:1229, 1985), disks are cultured on selective media, followed by shoot formation in about 2-4 weeks. Shoots that develop are excised from calli and transplanted to appropriate root-inducing selective medium. Appropriate selection media are known in the art (see, e.g., Curry and Cassells in: Plant Cell Culture Protocols, pp. 31-43, Humana Press, Totowa, N.J., 1999; Blackwell et al, IBID 19-30, 1999; Franklin and Dixon in: Plant Cell Culture, pp. 1-25, IRL Press, Oxford, 1994). Rooted plantlets are transplanted to soil as soon as possible after roots appear. The plantlets can be repotted, as required, until reaching maturity.

Regeneration also occurs from plant callus, tissues, organs, or parts. Transformation can be performed in the context of organ or plant part regeneration (see, e.g., Methods in Enzymology, vol. 118, and Klee, et al., Ann. Rev. Plant Phys., vol. 38:467, 1987). Utilizing a leaf disk-transformation-regeneration method (see, e.g., Horsch, et al., Science, vol. 227:1229, 1985), disks are cultured on selective media, followed by shoot formation in about 2-4 weeks. Shoots that develop are excised from calli and transplanted to appropriate root-inducing selective medium. Appropriate selection media are known in the art (see, e.g., Curry and Cassells in: Plant Cell Culture Protocols, pp. 31-43, Humana Press, Totowa, N.J., 1999; Blackwell et al, IBID 19-30, 1999; Franklin and Dixon in: Plant Cell Culture, pp. 1-25, IRL Press, Oxford, 1994). Rooted plantlets are transplanted to soil as soon as possible after roots appear. The plantlets can be repotted, as required, until reaching maturity.

Parts obtained from the transgenic plant, such as flowers, seeds, leaves, branches, fruit, and the like, are included in the invention. As will be appreciated, in some vegetatively propagated plant species, the root portion may be transgenic (i.e., be engineered to contain a nucleic acid molecule according to the invention), while the upper portion of the plant may not be. Alternatively, the portion of the plant grafted onto the root stock may be transgenic (i.e., be engineered to contain a nucleic acid molecule according to the invention), while the root stock may not be. In other embodiments, both the root stock and vegetatively propagated portions are transgenic. Progeny and variants, and mutants of the regenerated plants are also included within the scope of the present invention, provided that these parts comprise the introduced heterologous nucleic acid sequences.

B. Generation of Transgenic Plants With Desirable Traits

Transgenic plants according to the invention are resistant to, or tolerant of, biotic and abiotic stresses. Additionally, they exhibit delayed senescence.

Biotic stresses result directly or indirectly from a challenge by a biotic agent. Biotic agents include insects, fungi, bacteria, viruses, nematodes, viroids, mycloplasmas, etc. Biotic agents typically induce programmed cell death in affected plant cells. Such programmed cell death is thought to occur to inhibit the spread of an invading pathogen. However, the transgenic plants of the invention have exhibited resistance to a variety of biotic agents, including pathogens such as fungi and viruses. An exemplary pathogen is the fungal pathogen Sclerotinia sclerotiorum, which is one of the most nonspecific and omnivorous plant pathogens known. Further, a variety of other economically important pathogens are known, including the fungi Botrytis cinerea, Magnaportyhe grisea, Phytophthora spp, Cochliobolus spp, Fusarium graminearum and other Fusarium spp, nemtodes (such as the Meloidogyne, or “root knot”, nematodes), viruses such as tobacco mosaic virus (TMV) and tomato spotted wilt virus (TSWV), tobacco etch virus (TEV), tobacco necrosis virus (TNV), wheat streak mosaic virus (WSMV), soil borne wheat mosaic virus (SBWMV), barley yellow dwarf virus (BYDV), bacteria such as various Pseudomonas and Xanthomonas species, as well as many others.

Abiotic stress can be caused, for example, various environmental factors, such as drought, flooding) nutrient deficiency, radiation levels, air pollution, heat shock, cold shock, and soil toxicity, as well as herbicide damage, pesticide damage, or other agricultural practices. Accordingly, given that such abiotic agents play an increasing role in the viability of a variety of plant types including, food crops and ornamentals, the present invention can be utilized to produce plants or plant products (e.g., fruits, vegetables, seeds, flowers, etc.) with increased resistance to stresses such as these. Indeed, transgenic plants and plant products according to the invention are resistant to, or tolerant of, a plurality of such stresses, whether encountered simultaneously or at different times. As a result, the transgenic plants of the invention may be cultivated in new areas, thereby increasing the growth range for particular species or variety. In addition, because the transgenic plants of the invention are more tolerant to the range of growth conditions encountered in the cultivation of commercially relevant plant varieties, fewer plant varieties may be required over an existing, or even increase growth range. Similarly, improved stress resistance and tolerance will lead to increased yields of desired plant products under a variety of conditions.

One skilled in the art will readily recognize that given the disclosure provided herein, resistance to a particular biotic or abiotic stress, or combination of stresses, can be easily tested using whole plant or leaf sections, as appropriate. For example, a plant leaf may be inoculated with virus and lesion development and expansion may be measured at different time intervals. In another example, whole transgenic plants may be subjected to an abiotic stress such as high or low temperature. Stress responses, survival rates, etc. may be measured and compared to wild-type controls.

Senescence in plants is known to be a regulated process that ultimately results in cell death. Further, it is accompanied by many biochemical and structural changes, such as induction of cysteine proteases, RNases, etc., consistent with PCD. Inhibiting or delaying senescence can lead to longer shelf-lives for plant products, including fruits, vegetables, and flowers, as well as leading to increased longevity and aesthetic appeal of cut flowers and other ornamentals. In addition, in living plants increased flowering duration and fruit production may be achieved. Accordingly, the present invention has wide utility in both the food stuff market as well as the ornamental market.

Any known method for assessing senescence in plants or plant cells, tissues, or products may be used to test for decreased or delayed senescence. Such methods include, for example, characterization of fruit ripening processes, measurement of flower life, and detection of ethylene production (see, e.g., U.S. Pat. No. 5,702,933; Ryu, et al., Proc. Natl. Acad. Sci. USA, vol. 94:12717-21, 1997).
4. Methods of Modulating Apoptosis

The invention also provides methods for modulating apoptosis in a plant. Generally, such methods comprise generating a transgenic plant according to the invention and then identifying a transformed plant that, as compared to a wild-type plant of the same variety, exhibits an altered apoptotic response upon exposure to a biotic or abiotic stress, or combination of stresses. Any known method for assaying apoptosis may be used in this regard. For instance, a transformed plant or a portion thereof the plant may be challenged with a biotic or abiotic agent, after which the morphology of the inoculation site can observed for apoptotic signs. Alternatively, or in addition, cells or tissue from the inoculation site(s), as well as surrounding cells and tissues, if desired, can be further characterized by subsequent analysis for DNA fragmentation (e.g., by agarose gel electrophoresis), nuclear condensation (e.g., by Hoechst or DAPI staining), the change of the number of TUNEL-positive cells compared to control samples, etc.

The invention will be better understood by reference to the following Examples, which are intended to merely illustrate the best mode now known for practicing the invention. The scope of the invention is not to be considered limited thereto.

### EXAMPLES

### Example 1

### A Representative Stress Assay

This example describes a yeast-based assay for assessing STS sequences for stress protection efficacy. Here, a DNA molecule encoding a stress protection sequence (STS) is cloned into a plasmid suitable for replication and maintenance in Saccharomyces cerevisiae such as p427TEF (Dualsystems Biotech). Preferably, the STS-encoding sequence is cloned such that it is transcribed from a promoter sequence situated upstream (5′) of the STS sequence. A transcription terminator sequence preferably follows the STS-encoding sequence. A control plasmid lacking the STS-encoding sequence can also be made.

The resulting plasmids can be transformed into yeast using any suitable protocol, for example, the protocol described by Gietz and Schiestl (Gietz and Schiestl, 2007, Nat Protoc 2(1): 31-4). The resulting yeast cells carrying the plasmid containing the STS sequence can then be assayed for stress protection using a suitable assay. A description of one such assay is described in the following paragraphs.

Yeast cells are grown in rich nutrient medium such as YPD to an optical density (OD) of 0.5 measured at 600 nanometers (OD₆₀₀). Cells are then diluted 10-fold into fresh YPD medium containing hydrogen peroxide at increasing concentrations ranging from 1 to 5 mM. Cells are incubated at 30° C. for 12-48 hrs. At periodic intervals, such as every 4 hours, an aliquot is removed and the OD₆₀₀ of the cell culture read. The optical density of the STS plasmid-containing cultures versus the control cultures with plasmid lacking STS sequence is then plotted. Stress protection can also be measured by counting cells microscopically using a hemacytomer or counting colony forming units by plating an appropriate amount onto an agar plate and incubating at 30° C. for 2-3 days. The number of colonies growing on a plate is indicative of the number of viable cells in the culture at the time of sampling. Sequences which protect against stress will show higher growth compared to control cultures lacking the gene as measured by optical density at 600 nanometers, total viable cells, and/or the number of colony forming units obtained from agar medium plating.

### Example 2

### STS Confers Peroxide Stress Protection

This example describes the use of stress protection sequence (STS) according to the invention, designated iDi-176, to enhance cellular protection against stress caused during the culture of yeast by the addition to the medium of a reactive oxygen species, hydrogen peroxide. Here, a DNA molecule including the nucleotide sequence represented by SID 4 encoded the protective RNA species. The plasmid (pGilda backbone) harboring the expression cassette encoding the protective RNA molecule is depicted in Figure A, and was designated pGilda:Tef:Lex:176. As shown in the figure, the STS was flanked on its 5′ end by a peptide coding sequence from the LexA gene, and flanked on its 3′ end by sequence from a multiple cloning site present in the vector followed by an ADH1 terminator sequence. Expression of the RNA was driven by a TEF1 promoter. A vector, designated pGilda:Tef:Lex:Empty, containing all elements but that encoding the STS was generated for use as a acontrol.

Following construction of pGilda:Tef:Lex:176 and pGilda:Tef:Lex:Empty, each of the vectors was transformed into Saccharomyces cerevisiae strain InvSc1 by standard procedures to create the test and control strains InvSc1_pGilda:Tef:Lex:176 and InvSc1_pGilda:Tef:Lex:Empty, respectively. Eight replicates of each of the test and control strains were then tested by inoculating 5 mL overnight cultures from glycerol stocks stored at −80° C. The growth medium was synthetic dropout media minus histidine plus 1% glucose (SD−his+glu), as the pGilda vector plasmid carries the HIS auxotrophy gene for plasmid selection. The following morning the optical densities were spectrophotometrically measured at 600 nm (OD₆₀₀), after which each of the cultures were adjusted to an optical density OD₆₀₀=1.0 by diluting with an appropriate amount of fresh culture medium. Here, 3.16 mL of fresh growth medium was added to the InvSc1_pGilda:Tef:Lex:Empty overnight culture, and 9.05 mL of fresh culture medium was added to the InvSc1_pGilda:Tef:Lex:176 culture.

The test and control strains were then exposed to peroxide stress, as follows. For each of InvSc1_pGilda:Tef:Lex:Empty and InvSc1_pGilda:Tef:Lex:176, diluted overnight cultures were tested by adding 1.0 ml of the diluted, OD₆₀₀=1 overnight culture to each of eight 250 mL shake flasks containing 19 mL of stress medium, SD-his,glu_{—}3 mM H₂O₂, per flask. The stress medium was freshly prepared by adding 1.5 ml 3% H₂O₂ to 500 ml of SD−his+glu with gentle mixing to yield a 3% final H₂O₂ concentration. Peroxide concentration was confirmed by a standard peroxide assay. Following the addition of the cultures to the stress medium, the flasks were transferred to 28° C. Lab line shaker and shaken at 200 rpm. Peroxide concentrations and viable cell counts were determined for each flask at 6 hr intervals. To measure peroxide concentrations, an aliquot was taken from each flask for testing with a Aqua fast H₂O₂ strip to gauge peroxide concentration. H₂O₂ concentrations were then measured using a perox-O-quant assay as described by the manufacturer (PeroXOquant, Pierce, Rockford Ill., 61105). At each time point the OD₆₀₀ of each culture was recorded and viable cells were counted by plating cells on agar plates and incubating for 3 days at 30° C., followed by counting yeast colony forming units per milliliter of culture (cfu/ml).

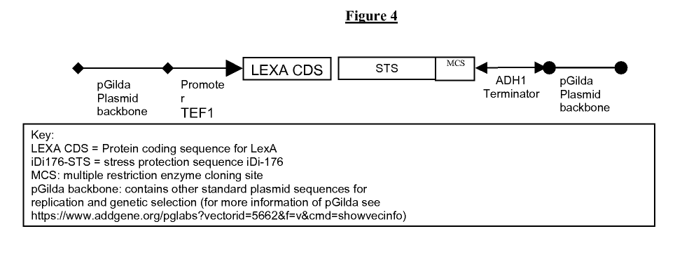

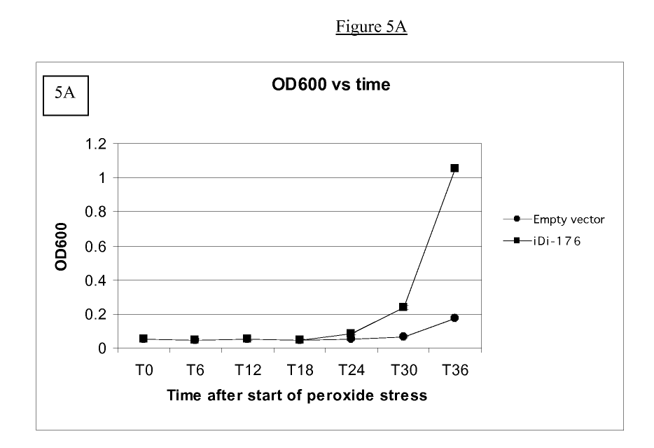

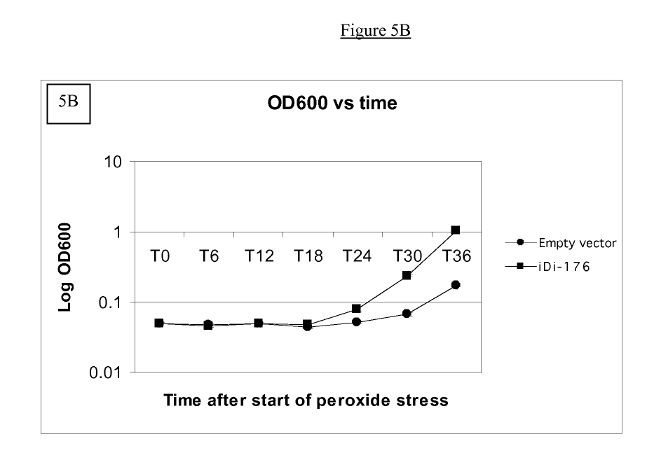

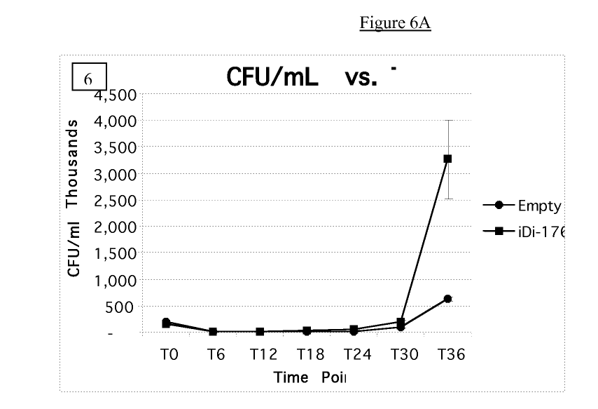

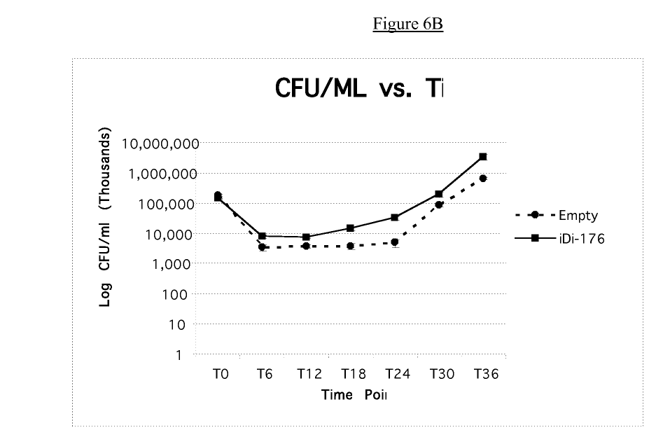

These experiments showed that the stress protective sequence iDi-176 enhanced cell survival and recovery from cytotoxic hydrogen peroxide stress by a factor of up to 19 fold at 36 hours after initiation of H₂O₂ stress. Statistically significant protection was seen as early as 24 hours for OD₆₀₀ assay (see FIG. 4B) and as early as 6 hours after treatment of cells with hydrogen peroxide when measuring viable cells using the cfu/ml assay, as shown in FIGS. 5A and 5B.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, it will be apparent that various genetic constructs can be generated that will encode a non-coding RNA molecule according to the invention and that will achieve the same or similar results. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An isolated nucleic acid molecule that encodes a non-coding RNA molecule that, upon expression in a eukaryotic cell, confers resistance and/or tolerance to one or more biotic and/or abiotic stresses, wherein the non-coding RNA molecule comprises a nucleotide sequence 5′-UUAUUUA-3′.

2. An expression cassette comprising a promoter operably associated with an isolated nucleic acid molecule according to claim 1.

3. A vector comprising an expression cassette according to claim 2.

4. A vector according to claim 3 further comprising a second nucleic acid molecule that encodes an expression product that confers a second desired trait.

5. A vector according to claim 2 wherein the promoter is selected from the group consisting of a constitutive promoter and an inducible promoter.

6. A host cell transformed with a vector according to claim 5.

7. A host cell according to claim 6 selected from the group consisting of a mammalian cell, a yeast cell, a plant cell, and a bacterial cell.

8. A transgenic plant cell that includes an expression cassette according to claim 2.

9. A transgenic plant that includes at least one cell stably transformed with an expression cassette according to claim 2.

10. A transgenic plant according to claim 9 that exhibits increased stress resistance when cultivated under stressful conditions, as compared to a wild-type plant of the same variety as the transgenic plant.

11. A transgenic plant according to claim 9 having at least one tissue that exhibits reduced senescence, as compared to the same tissue(s) of a wild-type plant of the same variety as the transgenic plant.

12. A transgenic plant according to claim 9 that is a plant selected from the group consisting of a tomato, potato, arabidopsis, tobacco, cotton, rapeseed, field bean, soybean, pepper, lettuce, pea, alfalfa, clover, cole, cabbage, broccoli, cauliflower, Brussels sprout, radish, carrot, beet, eggplant, spinach, cucumber, squash, melon, cantaloupe, sunflower, ornamental, asparagus, corn, barley, wheat, rice, sorghum, onion, pearl millet, rye, and oat plant.

13. A method of producing a transgenic plant cell, comprising transforming a plant cell with a nucleic acid molecule according to claim 1.

14. A method of generating a transgenic plant, comprising producing a transgenic plant cell according to claim 13 from which a transgenic plant is then generated.

15. A method of plant cultivation, comprising cultivating a transgenic plant according to claim 9.

16. A method according to claim 15, wherein the cultivation occurs in an environment where the transgenic plant may be exposed under anticipated conditions to a stress which, in the absence of expression of one or more non-coding RNA molecules from the the nucleic acid molecule, would result in injury to or death of the plant.

17. A method according to claim 16 wherein the stress is selected from the group consisting of an abiotic stress and a biotic stress.

18. A method according to claim 15 wherein the transgenic plant is selected from the group consisting of a transgenic tomato, potato, arabidopsis, tobacco, cotton, rapeseed, field bean, soybean, pepper, lettuce, pea, alfalfa, clover, cole, cabbage, broccoli, cauliflower, Brussels sprout, radish, carrot, beet, eggplant, spinach, cucumber, squash, melon, cantaloupe, sunflower, ornamental, asparagus, corn, barley, wheat, rice, sorghum, onion, pearl millet, rye, and oat plant.

19. A transgenic yeast cell that includes an expression cassette according to claim 2.
